(19) United States
(12) Patent Application Publication
(10) Pub. No.: US 2006/0231832 A1
(43) Pub. Date: Oct. 19, 2006

(54) Liquid crystalline organic compound, organic semiconductor structure, organic semiconductor device, and process for producing liquid crystalline organic compound

(76) Inventors: Keiji Tokunaga, Tokyo-to (JP); Yudai Yamashita, Tokyo-to (JP); Jun-ichi Hanna, Yokohama-shi (JP)

Correspondence Address: LADAS & PARRY LLP, 224 SOUTH MICHIGAN AVENUE, SUITE 1600, CHICAGO, IL, 60604, US

(21) Appl. No.: 11/370,779

(22) Filed: Mar. 8, 2006

(30) Foreign Application Priority Data
Mar. 9, 2005 (JP) 2005-066202

(51) Int. Cl. H01L 29/08

(52) U.S. Cl. 257/40

(57) ABSTRACT

A liquid crystalline organic compound which gives a liquid crystal phase in a temperature range containing at least a room temperature and can exhibit a high charge mobility; an organic semiconductor structure and an organic semiconductor device each having the liquid crystalline organic compound are provided.
The liquid crystalline organic compound contains any one of benzothiazole, benzoselenazole, benzoxazole and indene skeletons represented by the following chemical formula 1: 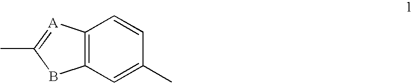 wherein A is a nitrogen atom or a CH group, and B is a sulfur, selenium or oxygen atom is contained as Z1 in the following chemical formula 2:
R1-Y1-Z1-Y2-R2 2
wherein, R1 and R2 are each independently a saturated or unsaturated hydrocarbon of a straight chain, a branched chain or a cyclic structure having 1 to 22 carbon atoms; R1 and R2 may be each independently bonded directly to Z1 without interposing Y1 or Y2 therebetween; and Y1 and Y2 are each independently selected from the group consisting of oxygen and selenium atoms and —CO—, —OCO—, —COO—, —N═CH—, —CONH—, —NH—, —NHCOO and —CH₂ groups.

### BACKGROUND OF THE INVENTION

1. Field of the Invention

The present invention relates to a liquid crystalline organic compound which gives a liquid crystal phase in a temperature range containing at least a room temperature and can exhibit a high charge mobility; an organic semiconductor structure and an organic semiconductor device each having the liquid crystalline organic compound; and a process for producing a liquid crystalline organic compound. The invention relates in particular to a liquid crystalline organic compound that can be continuously produced with uniform performances over a sufficiently wide area; and others.

2. Description of the Related Art

Conventionally, as liquid crystalline organic compounds, ones having various structures have been known, and have widely been used mainly as materials for liquid crystal displays. A typical example of an element constituting an organic semiconductor device is a thin film transistor (may also be referred to as an organic TFT) in which an organic semiconductor is used in an active layer (hereinafter referred to as an organic semiconductor layer). In this thin transistor, the organic semiconductor layer is formed by vacuum film-forming molecular crystal of a compound, a typical example of which is a pentacene. It is reported that according to a process for forming an organic semiconductor layer by vacuum film-formation, an organic semiconductor layer having a high charge mobility over 1.0 cm²/V·s can be obtained by optimizing conditions for the film-formation (see, for example, Y. -Y. Lin, D. J. Gundlach, S. Nelson, and T. N. Jackson, “Stacked Pentacene Layer Organic Thin-Film Transistors with Improved Characteristics”, IEEE Electron Device Lett. 606, 18 (1997)).

However, the organic semiconductor layer formed by the above-mentioned vacuum film-formation generally exhibits a polycrystalline state that microcrystals gather, and thus a large number of grain boundaries are liable to be present. Moreover, defects are easily generated. The grain boundaries or defects hinder the transportation of charges. Accordingly, in the case of forming an organic semiconductor layer, which is an element constituting an organic semiconductor device, by the vacuum film-formation, it is in reality difficult to form the organic semiconductor layer continuously with uniform performances over a sufficiently wide area.

As a material exhibiting a high charge mobility, a discotic liquid crystal is known (see, for example, D. Adam, F. Closss, T. Frey, D. Funhoff, D. Haarer, H. Ringsdorf, P. Schunaher, and K. Siemensmyer, Phys. Rev. Lett., 457, 70 (1993)). However, in this discotic liquid crystal, a charge is transported based on an one-dimensional charge transporting mechanism along the columnar molecular alignment thereof; therefore, strict control over the molecular alignment is required so as to cause a problem that the liquid crystal is not industrially used at ease. A successive example of a thin film transistor in which this discotic liquid crystal is used as a material constituting an organic semiconductor layer has not yet been reported.

It has already been reported that a rodlike liquid crystalline material such as a phenylbenzothiazole derivative also exhibits a high charge mobility in a liquid crystal state (see, for example, M. Funahashi and J. Hanna, Jpn. J. Appl. Phys., 35, L703-L705, and Japanese Patent Application Laid-Open No. 9-59266). However, there are problems such that when the 6-position of phenylbenzothiazole is brominated in the stage of a synthesis route of the phenylbenzothiazole derivative, the 3′-position of the phenylbenzothiazole derivative is also brominated so that the derivative is not easily purified; a sulfur atom is interposed as a spacer moiety between the core and the terminal of the structure thereof but the carbon-sulfur bond is easily oxidized and is not necessarily chemically stable; and the temperature range for keeping the state of liquid crystal is a narrow range of 90 to 100° C. Thus, a successful example of a thin film transistor in which a rodlike liquid crystalline material is used in an organic semiconductor layer has not yet been reported.

Rodlike liquid crystalline materials exhibit various liquid crystal states, but the charge mobility thereof tends to become higher as the structure regularity of the liquid crystalline materials becomes higher. However, when the liquid crystalline materials are transited into a crystal state having a higher structure regularity, the mobility of charges is conversely lowered or is not observed. Thus, naturally, no performances of thin film transistors are exhibited.

In order to use a rodlike liquid crystalline material in the state of a liquid crystal exhibiting a charge mobility, it is necessary to seal the material into a glass cell or the like. Thus, there are restrictions for the production of a device therefor. Furthermore, the temperature at which the rodlike liquid crystalline material exhibits a high liquid crystal property is a relatively high temperature, and thus the rodlike liquid crystalline material cannot be used at liquid-crystal-state keeping temperatures which contain a temperature of around a room temperature.

In the case of using a molecule-dispersion type polymer material as a liquid crystalline organic compound, an organic semiconductor layer having a uniform charge-mobility property over a large area can be formed by coating the liquid crystalline organic compound. However, the formed organic semiconductor layer has a low charge mobility of 1.0×10⁻⁵ to 1.0×10⁻⁶ cm²/V·s and further there remain problems that the mobility has the temperature dependency or the electric field dependency.

In order to solve the above-mentioned problems, the present invention has been made, and an object thereof is to provide a liquid crystalline organic compound which gives a liquid crystal phase in a temperature range containing at least a room temperature and can exhibit a high charge mobility, and an organic semiconductor structure and an organic semiconductor device which each have this liquid crystalline organic compound. Another object of the invention is to provide a process for producing such a liquid crystalline organic compound effectively.

### SUMMARY OF THE INVENTION

The liquid crystalline organic compound according to a first aspect of the present invention for attaining the objects is a liquid crystalline organic compound, wherein any one of benzothiazole, benzoselenazole, benzoxazole and indene skeletons represented by the following chemical formula 1: 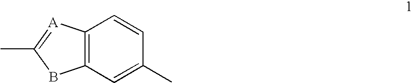
in which A is a nitrogen atom or a CH group, and B is a sulfur, selenium or oxygen atom, is contained as Z1 in the following chemical formula 2:
R1-Y1-Z1-Y2-R2 2
in which R1 and R2 are each independently a saturated or unsaturated hydrocarbon of a straight chain, a branched chain or a cyclic structure having 1 to 22 carbon atoms; R1 and R2 may be each independently bonded directly to Z1 without interposing Y1 or Y2 therebetween; and Y1 and Y2 are each independently selected from the group consisting of oxygen and selenium atoms and —CO—, —OCO—, —COO—, —N═CH—, —CONH—, —NH—, —NHCOO and —CH₂ groups.

The liquid crystalline organic compound of this aspect gives a liquid crystal phase in a wide temperature range containing at least a room temperature and can exhibit a high charge mobility.

The liquid crystalline organic compound according to a second aspect of the invention for attaining the above-mentioned objects is a liquid crystalline organic compound, wherein any one of benzothiazole, benzoselenazole, benzoxazole and indene skeletons represented by the following chemical formula 1: 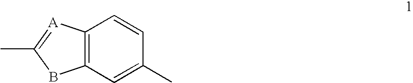
in which A is a nitrogen atom or a CH group, and B is a sulfur, selenium or oxygen atom, is contained as each of Z1 and Z2 in the following chemical formula 3:
R1-Y1-Z1-X-Z2-Y2-R2 3
in which R1 and R2 are each independently a saturated or unsaturated hydrocarbon of a straight chain, a branched chain or a cyclic structure having 1 to 22 carbon atoms; R1 may be bonded directly to Z1 without interposing Y1 therebetween and R2 may be bonded directly to Z2 without interposing Y2 therebetween; Y1 and Y2 are each independently selected from the group consisting of oxygen and selenium atoms and —CO—, —OCO—, —COO—, —N═CH—, —CONH—, —NH—, —NHCOO and —CH₂groups; and X may be a saturated or unsaturated hydrocarbon having a straight chain, a branched chain or a cyclic structure having 1 to 22 carbon atoms, or X may not be present so as to bond Z1 directly to Z2.

The liquid crystalline organic compound of this aspect gives a liquid crystal phase in a wide temperature range containing at least a room temperature and can exhibit a high charge mobility.

The liquid crystalline organic compound of the invention according to the first or second aspect preferably exhibits at least one kind of smectic liquid crystal phase state at the pyrolysis temperature thereof or lower.

According to this embodiment, a molecular alignment thin film uniform and flexible over a wide area can be formed since this embodiment can exhibit at least one kind of smectic liquid crystal phase state at the pyrolysis temperature thereof or lower.

The organic semiconductor structure of the present invention for attaining the above-mentioned objects is an organic semiconductor structure having an organic semiconductor layer comprising the liquid crystalline organic compound according to the invention, wherein the liquid crystalline organic compound exhibits at least one kind of smectic liquid crystal phase state at the pyrolysis temperature thereof or lower, and the organic semiconductor layer has an electron mobility of 1.0×10⁻⁵ cm²/V·s or more, or a hole transporting mobility of 1.0×10⁻⁵ cm²/V·s or more.

According to this aspect, a molecular alignment thin film uniform and flexible over a wide area can be formed since the liquid crystalline organic compound for forming the organic semiconductor layer exhibits at least one kind of smectic liquid crystal phase state at the pyrolysis temperature thereof or lower. Consequently, the structure can be applied to a thin film transistor which can be used in a flexible display device or the like, or to other devices.

The organic semiconductor device of the invention for attaining the objects is an organic semiconductor device comprising at least a substrate, a gate electrode, a gate insulating layer, an organic semiconductor layer, a drain electrode, and a source electrode, wherein the organic semiconductor layer is formed so as to comprise the liquid crystalline organic compound according to any one of the first to third aspects.

The process for producing a liquid crystalline organic compound of the invention, for attaining the objects, is a process for producing the liquid crystalline organic compound of the invention comprising: a step of synthesizing a crude liquid crystalline organic compound; and a step of using at least two kinds of solvents selected from the group consisting of a protonic polar solvent, an aprotonic polar solvent, a basic solvent, a halogenated hydrocarbon solvent, and a nonpolar solvent to purify the synthesized crude liquid crystalline organic compound by a recrystallization method.

According to this aspect, impurities, which are causes of a fall in the charge mobility, can be effectively removed since the synthesized crude liquid crystalline organic compound is purified by the recrystallization method using at least two kinds of solvents selected from a protonic polar solvent, an aprotonic polar solvent, a basic solvent, a halogenated hydrocarbon solvent, and a nonpolar solvent.

The liquid crystalline organic compound of the invention has a chemical structure having a liquid crystal core made of any one of the benzothiazole, benzoselenazole, benzoxazole and indene skeletons. These liquid crystalline organic compounds make it possible to give a liquid crystal phase in a temperature range containing at least a room temperature and further exhibit a high charge mobility.

According to the organic semiconductor structure and the organic semiconductor device of the invention, the organic semiconductor layer thereof can be made of the liquid crystalline organic compound of the invention. Accordingly, an organic semiconductor thin film uniform and flexible over a wide area can be formed, and the structure and the device can be applied to a thin film transistor which can be used in a flexible display device or the like, or other devices.

According to the process for producing a liquid crystalline organic compound of the invention, impurities, which are causes of a fall in the charge mobility, can be effectively removed. Therefore, the liquid crystal compound, which gives a liquid crystal phase in a temperature range containing at least a room temperature and can exhibit a high charge mobility, can be effectively produced.

### BRIEF DESCRIPTION OF THE DRAWINGS

FIG. 1 is a model diagram showing an example of an organic semiconductor layer in which a liquid crystalline organic compound of the invention is self-organized.

FIG. 2 is a sectional view showing an example of the organic semiconductor device of the invention.

FIG. 3 is an example of a block diagram for transient photocurrent measurement (the TOF (Time Of Flight) method).

### DETAILED DESCRIPTION OF THE PREFERRED EMBODIMENT

Hereinafter, a liquid crystalline organic compound and a process for producing the same of the invention will be described in detail.

(Liquid Crystalline Organic Compound)

The liquid crystalline organic compound of the first aspect of the invention is a liquid crystalline organic compound, wherein anyone of benzothiazole, benzoselenazole, benzoxazole and indene skeletons represented by the above-mentioned chemical formula 1, in which A is a nitrogen atom or a CH group, and B is a sulfur, selenium or oxygen atom, is contained as Z1 in the above-mentioned chemical formula 2.

In the liquid crystalline organic compound according to the first aspect, R1 and R2 in the chemical formula 2 are each independently a saturated or unsaturated hydrocarbon of a straight chain, a branched chain or a cyclic structure having 1 to 22 carbon atoms; R1 may be bonded through Y1 to Zl, and/or R2 may be bonded through Y2 to Zl; R1 and R2 may be bonded directly to Z1 without interposing Y1 or Y2 therebetween; and Y1 and Y2 are each independently selected from the group consisting of oxygen and selenium atoms and —CO—, —OCO—, —COO—, —N═CH—, —CONH—, —NH—, —NHCOO and —CH₂ groups.

The liquid crystalline organic compound of the second aspect of the invention is a liquid crystalline organic compound, in which any one of benzothiazole, benzoselenazole, benzoxazole and indene skeletons represented by the chemical formula 1, wherein A is a nitrogen atom or a CH group, and B is a sulfur, selenium or oxygen atom, is contained as each of Z1 and Z2 in the following chemical formula 3.

In the liquid crystalline organic compound according to the second aspect, R1 and R2 in the chemical formula 3 are each independently a saturated or unsaturated hydrocarbon of a straight chain, a branched chain or a cyclic structure having 1 to 22 carbon atoms; R1 may be bonded through Y1 to Z1, and/or R2 may be bonded through Y2 to Z2; R1 and R2 may be bonded directly to Z1 and Z2 without interposing Y1 and Y2 therebetween; Y1 and Y2 are each independently selected from the group consisting of oxygen and selenium atoms and —CO—, —OCO—, —COO—, —N═CH—, —CONH—, —NH—, —NHCOO and —CH₂ groups; and X may be a saturated or unsaturated hydrocarbon of a straight chain, a branched chain or a cyclic structure having 1 to 22 carbon atoms, or X may not be present so as to bond Z1 directly to Z2.

In the liquid crystalline organic compounds according to the first and second aspects, R1 and R2 are each preferably a saturated or unsaturated hydrocarbon of a straight chain, a branched chain or a cyclic structure having 3 to 15 carbon atoms. The liquid crystalline organic compounds comprising R1 and R2 having 3 to 15 carbon atoms have an advantage that the compounds easily give a liquid crystal phase and can make the temperature range for the liquid-crystal-state keeping temperature wide.

Specific examples of the saturated or unsaturated hydrocarbon, which has a straight chain, a branched chain or a cyclic structure having 1 to 22 carbon atoms, include methyl, ethyl, propyl, isopropyl, butyl, isobutyl, tertiary-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, eicosyl, henicosyl, heneicosyl, docosyl, ethene, propene, butene, pentene, hexene, heptene, octene, nonene, decene, undecene, dodecene, tridecene, tetradecene, pentadecene, hexadecene, heptadecene, octadecene, nonadecene, icosene, eicosene, henicosene, heneicosene, docosene, ethyne, propyne, butyne, pentyne, hexyne, heptyne, octyne, nonyne, decyne, undecyne, dodecyne, tridecyne, tetradecyne, pentadecyne, hexadecyne, heptadecyne, octadecyne, nonadecyne, icosyne, eicosyne, henicosyne, heneicosyne, docosyne, cyclopentyl, cyclohexyl, and cycloheptyl. Specific examples of the preferably-used saturated or unsaturated hydrocarbon, which has a straight chain, a branched chain or a cyclic structure having 3 to 15 carbon atoms, include propyl, isopropyl, butyl, isobutyl, tertiary-butyl, pentyl, hexyl, heptyl, octyl, nonyl, decyl, undecyl, dodecyl, tridecyl, tetradecyl, pentadecyl, pentene, hexene, heptene, octene, nonene, decene, undecene, dodecene, tridecene, propyne, butyne, pentyne, hexyne, heptyne, octyne, nonyne, decyne, undecyne, dodecyne, tridecyne, tetradecyne, pentadecyne, cyclopentyl, cyclohexyl, and cycloheptyl.

In the liquid crystalline organic compounds according to the first and second aspects described above, it is preferred from the viewpoint of chemical stability that Y1 and Y2 are each independently selected from an oxygen atom, and —CO—, —OCO—, —COO—, —N═CH—, —CONH—, —NH—, —NHCOO and —CH₂ groups. It is more preferred that Y1 and Y2 are each independently selected from an oxygen atom, and —CO—, —OCO—, —COO—, and —CH₂ groups. Among conventional liquid crystalline organic compounds, compounds wherein either of Y1 or Y2 is a sulfur atom are known. However, in such compounds, the carbon-sulfur bond is easily oxidized so that the compounds are chemically unstable. Thereafter, therefore, the compounds may not easily be purified. Accordingly, in the liquid crystalline organic compound of the invention, it is preferred that neither Y1 nor Y2 is a sulfur atom.

The liquid crystalline organic compound of the invention exhibits at least one kind of smectic liquid crystal phase state at the pyrolysis temperature thereof or lower. The wording “the pyrolysis temperature thereof or lower” means temperatures at which the liquid crystalline organic compound itself is not thermally decomposed. Specifically, the liquid crystalline organic compound of the invention gives a high-order liquid crystal phase in a temperature range containing at least a room temperature. The wording “a temperature range containing at least a room temperature” means a temperature range in which the liquid crystalline organic compound of the invention gives a liquid crystal phase. For example, as liquid crystalline organic compounds in respective examples which will be described later, there are described: a liquid crystalline organic compound in which the lower limit of the above-mentioned temperature range is about −50° C.; and a liquid crystalline organic compound in which the upper limit of the temperature range is about 100° C. The pyrolysis temperature is varied in accordance with the particular liquid crystalline organic compounds contained in the technical scope of the invention. The wording “exhibits at least one kind of smectic liquid crystal phase state” means that when the liquid crystalline organic compound of the invention is used at the pyrolysis temperature or lower (that is, in a temperature range containing at least a room temperature), the compound can exhibit plural kinds of liquid crystal phase states or at lowest one kind of liquid crystal phase state. In the case of, for example, a smectic (this wording being abbreviated hereinafter to “Sm” according to circumferences) liquid crystal phase, plural kinds of liquid crystal states of SmA, SmB and SmC phases and other phases are known. The wording “exhibits at least one kind of smectic liquid crystal phase state” means that at least one kind of liquid crystal state out of these liquid crystal states is exhibited within the above-mentioned temperature range.

Since the liquid crystalline organic compound of the invention exhibits at least one kind of smectic liquid crystal phase state at the pyrolysis temperature thereof or lower (that is, the compound exhibits a high-order liquid crystal phase in a temperature range containing at least a room temperature), a molecular alignment thin film uniform and flexible over a wide area can be formed.

The smectic liquid crystal phase is preferably a rodlike smectic liquid crystal phase. Since liquid crystal has self-organization effect, molecular order is spontaneously formed at ease in the rodlike smectic liquid crystal phase so that a high molecular order, such as molecular crystal, is formed for a charge transfer path. When an organic semiconductor layer is made of a liquid crystalline organic compound exhibiting such a smectic liquid crystal phase, excellent charge transfer properties such as molecular crystal can be supplied into the organic semiconductor layer. It is particularly preferred that the organic semiconductor layer is made of a liquid crystalline organic compound which can exhibit a high-order smectic liquid crystal phase.

The organic semiconductor layer made of the liquid crystalline organic compound has a charge-mobility property that a high-speed charge transfer ability is exhibited about each of a high electron and hole based on hopping conduction. This would be based on the following reason.

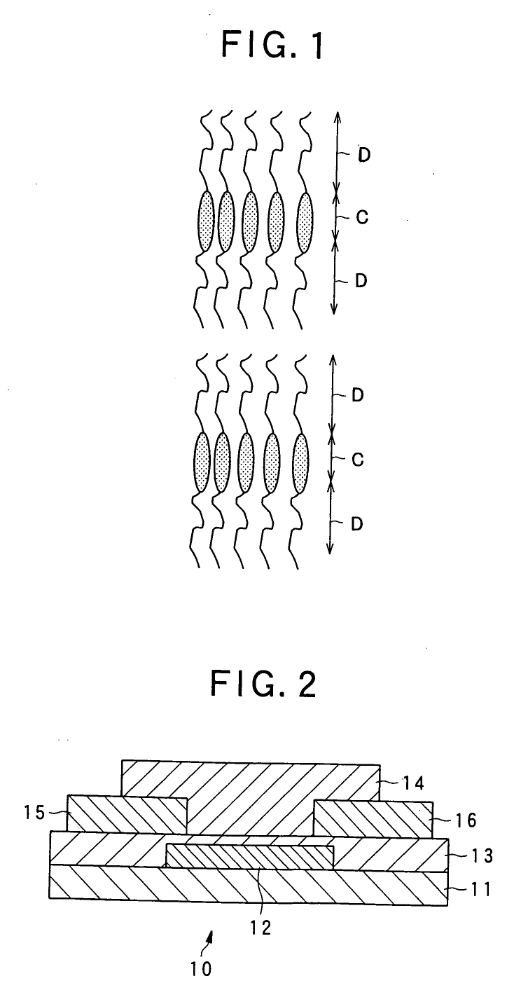

FIG. 1 is a model diagram showing an example of an organic semiconductor layer in which a liquid crystalline organic compound of the invention is self-organized. The liquid crystalline organic compound undergoes self-organization by the presence of R1 and R2, which are each a saturated or unsaturated hydrocarbon of a straight chain, a branched chain or a cyclic structure having 1 to 22 (preferably 3 to 15) carbon atoms (see the above-mentioned chemical formulas (2) and (3)), so that the compound is aligned very orderly. In the organic semiconductor layer, regions C are formed which are molecule-aggregated moieties having a high self-organization property by action of this liquid crystalline organic compound. In the regions C, rigid skeleton moieties (i.e., Z1 and Z2 in the chemical formulas 2 and 3) of the liquid crystalline organic compound are adjacent to neighboring liquid crystalline organic compounds at a very small distance. As a result, in the regions C of the liquid crystalline organic compound, 7 electron orbitals overlap largely with each other so that high-speed electron conduction and high-speed hole conduction are caused based on hopping conduction. Accordingly, the formed organic semiconductor layer exhibits a high charge transfer property. In the case, the interval between the skeletons is from about 0.3 to 0.5 nm.

The organic semiconductor layer made of the liquid crystalline organic compound of the invention has the regions C, wherein the rigid skeleton moieties of the liquid crystalline organic compound continuously overlap with each other over a considerable distance by the self-organization of the compound itself; accordingly, hopping conduction of electrons or holes is easily caused. In the case that no high molecular order is realized over a long distance as is seen in microcrystal, electrons or holes are trapped in crystal grain boundaries thereof, so that a high conductivity cannot be expected.

In the liquid crystalline organic compound which gives a smectic liquid crystal phase, R1 and R2 in the chemical formulas 2and3 form rich regions D. The regions partition off the moieties of the regions C, which become an electron transfer path, and function as buffer layers so as to produce an effect of causing exhibition of a large charge transfer anisotropy.

Preferred examples of the specific structure for the liquid crystalline organic compound of the invention are shown in Tables 1 and 2. However, the specific structure is not limited thereto. Table 1 shows examples of the structure of the compound 2, and Table 2 shows those of the structure of the compound 3.

TABLE 1

| R1 | Y1 | Z1 | Y2 | R2 |
|---|---|---|---|---|
| C₇H₁₅ | O | Chemical formula 4 | None | C₁₂H₂₅ |
| C₇H₁₅ | O | Chemical formula 6 | None | C₁₂H₂₅ |
| C₇H₁₅ | O | Chemical formula 8 | None | C₁₂H₂₅ |
| C₈H₁₇ | None | Chemical formula 4 | O | C₄H₉ |
| C₈H₁₇ | None | Chemical formula 6 | O | C₄H₉ |
| C₈H₁₇ | None | Chemical formula 8 | O | C₄H₉ |
| C₈H₁₇ | None | Chemical formula 4 | None | C₁₂H₂₅ |
| C₈H₁₇ | None | Chemical formula 6 | None | C₁₂H₂₅ |
| C₈H₁₇ | None | Chemical formula 8 | None | C₁₂H₂₅ |

Notes)
O represents an oxygen atom.

TABLE 2

| R1 | Y1 | Z1 | X | Z2 | Y2 | R2 |
|---|---|---|---|---|---|---|
| C₁₂H₂₅ | None | Chemical formula 5 | None | Chemical formula 4 | None | C₁₂H₂₅ |
| C₁₂H₂₅ | None | Chemical formula 7 | None | Chemical formula 6 | None | C₁₂H₂₅ |
| C₁₂H₂₅ | None | Chemical formula 9 | None | Chemical formula 8 | None | C₁₂H₂₅ |
| C₁₂H₂₅ | None | Chemical formula 5 | C₆H₄ | Chemical formula 4 | None | C₁₂H₂₅ |
| C₁₂H₂₅ | None | Chemical formula 7 | C₆H₄ | Chemical formula 6 | None | C₁₂H₂₅ |
| C₁₂H₂₅ | None | Chemical formula 9 | C₆H₄ | Chemical formula 8 | None | C₁₂H₂₅ |
| C₁₂H₂₅ | None | Chemical formula 5 | None | Chemical formula 6 | None | C₁₂H₂₅ |
| C₁₂H₂₅ | None | Chemical formula 5 | None | Chemical formula 8 | None | C₁₂H₂₅ |
| C₁₂H₂₅ | None | Chemical formula 9 | None | Chemical formula 6 | None | C₁₂H₂₅ |
| C₁₂H₂₅ | None | Chemical formula 5 | C₆H₄ | Chemical formula 6 | None | C₁₂H₂₅ |
| C₁₂H₂₅ | None | Chemical formula 5 | C₆H₄ | Chemical formula 8 | None | C₁₂H₂₅ |
| C₁₂H₂₅ | None | Chemical formula 9 | C₆H₄ | Chemical formula 6 | None | C₁₂H₂₅ |
| C₇H₁₅ | None | Chemical formula 4 | None | Chemical formula 4 | None | C₁₂H₂₅ |
| C₇H₁₅ | None | Chemical formula 6 | None | Chemical formula 6 | None | C₁₂H₂₅ |
| C₇H₁₅ | None | Chemical formula 8 | None | Chemical formula 8 | None | C₁₂H₂₅ |
| C₇H₁₅ | None | Chemical formula 4 | None | Chemical formula 5 | None | C₇H₁₅ |
| C₇H₁₅ | None | Chemical formula 6 | None | Chemical formula 7 | None | C₇H₁₅ |
| C₇H₁₅ | None | Chemical formula 8 | None | Chemical formula 9 | None | C₇H₁₅ |
| C₇H₁₅ | None | Chemical formula 4 | None | Chemical formula 7 | None | C₇H₁₅ |
| C₇H₁₅ | None | Chemical formula 4 | None | Chemical formula 9 | None | C₇H₁₅ |
| C₇H₁₅ | None | Chemical formula 6 | None | Chemical formula 9 | None | C₇H₁₅ |

Chemical formulas 4 to 9 in Tables 1 and 2 shown above are as follows.

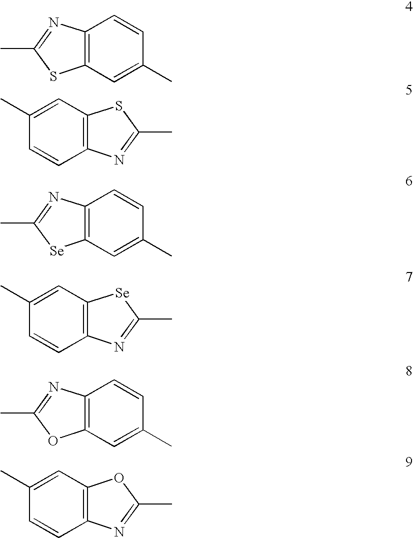

The liquid crystalline organic compound of the invention is selected, considering a property required for the organic semiconductor layer to be finally formed. About the property for the selection, the charge mobility of the electron or hole is at least 1.0×10⁻⁵ cm²/V·s or more, and is desirably 1.0×10⁻⁴ cm²/V·s or more. The charge mobility referred to herein means the moving speed of charges per second in a unit field, and can be measured by the Time Of Flight method (hereinafter referred to as the TOF method).

The liquid crystalline organic compound of the invention has a chemical structure having a liquid crystal core made of any one of the benzothiazole, benzoselenazole, benzoxazole and indene skeletons. This liquid crystalline organic compound makes it possible to give a liquid crystal phase in a temperature range containing at least a room temperature and further exhibit a high charge mobility.

(Organic Semiconductor Structure)

The organic semiconductor structure of the invention is an organic semiconductor structure which has an organic semiconductor layer comprising the above-mentioned liquid crystalline organic compound of the invention, wherein the liquid crystalline organic compound exhibits at least one kind of smectic liquid crystal phase state at the pyrolysis temperature thereof or lower, and the organic semiconductor layer has an electron mobility of 1.0×10⁻⁵ cm²/V·s or more, or a hole transporting mobility of 1.0×10⁻⁵ cm²/V·s or more.

The organic semiconductor layer is formed by aligning the liquid crystalline organic compound of the invention. The alignment can be attained by laminating the liquid crystalline organic compound onto a liquid crystal alignment layer made of, for example, a polyimide material, or laminating the compound onto a liquid crystal alignment layer having minute irregularities on the surface thereof and made of a curable resin.

The above-mentioned liquid crystalline organic compound has fluidity at not lower than temperatures at which the liquid crystal state thereof is kept. Thus, the compound can be coated in this state. According to this method, an organic semiconductor layer having an excellent charge transfer property and a large area can be very easily formed. As the coating method in this case, various coating methods and printing methods can be used.

Examples of the organic semiconductor structure of the invention include a first embodiment in which a liquid crystal alignment layer and an organic semiconductor layer are successively laminated onto a substrate, a second embodiment in which an organic semiconductor layer and a liquid crystal alignment layer are successively laminated onto a substrate, and a third embodiment in which a liquid crystal alignment layer, an organic semiconductor layer and a liquid crystal alignment layer are successively laminated onto a substrate. In the invention, its organic semiconductor layer is formed into a form that the layer contacts its layer subjected to alignment treatment, whereby a high alignment property can be given to the liquid crystal phase constituting the liquid crystalline organic compound.

As described above, the organic semiconductor structure of the invention contains an organic semiconductor layer comprising a liquid crystalline organic compound exhibiting a high-order liquid crystal phase in a temperature range containing at least a room temperature; therefore, the organic semiconductor layer can be used as a homogeneous molecular alignment thin film which keeps flexibility as liquid crystal. Since the organic semiconductor layer exhibits the high-order liquid crystal phase in the wide temperature range, a dense packing structure in a form near a molecular crystal phase can be realized and a high charge transfer property, desirably a charge mobility of 1.0×10⁻⁴ cm²/V·s or more, can be exhibited. As a result, a molecular alignment thin film uniform and flexible over a wide area can be formed, and the film can be applied to a thin film transistor which can be used in a flexible display or the like, or other devices.

(Organic Semiconductor Device)

FIG. 2 is a sectional view showing an example of the organic semiconductor device of the invention. The organic semiconductor device 10 according to the invention is fabricated to comprise at least a substrate 11, a gate electrode 12, a gate insulating layer 13, an organic semiconductor layer 14, a drain electrode 15 and a source electrode 16. In this organic semiconductor device 10, the organic semiconductor layer 14 is made of a liquid crystalline organic compound constituting the above-mentioned organic semiconductor structure of the invention.

An example of the structure of the organic semiconductor device 10 according to the invention is a reverse stagger structure in which the gate electrode 12, the gate insulating layer 13, the organic semiconductor layer 14 aligned, the drain electrode 15 and the source electrode 16, and a protective film 17 are formed, in this order, onto the substrate 11, or a coplanar structure in which the gate 12, the gate insulating layer 13, the drain electrode 15 and the source electrode 16, the organic semiconductor layer 14, and a protective film (not shown) are formed, in this order, onto the substrate 11. The organic semiconductor device 10 having such a structure works in a storage state or a depletion state in accordance with the polarity of voltage applied to the gate electrode 12.

Other aspects of the structure of the organic semiconductor device of the present invention include the following: (i) substrate/gate electrode/gate insulating layer (which also functions as a liquid crystal alignment layer)/source-drain electrodes/liquid crystalline organic semiconductor layer(/protective layer), (ii) substrate/gate electrode/gate insulating layer/source-drain electrodes/liquid crystal alignment layer/liquid crystalline organic semiconductor layer(/protective layer), (iii) substrate/gate electrode/gate insulating layer (which also functions as a liquid crystal alignment layer)/liquid crystalline organic semiconductor layer/source-drain electrodes/(protective layer), (iv) substrate/gate electrode/gate insulating layer (which also functions as a liquid crystal alignment layer)/liquid crystalline organic semiconductor layer/substrate in which source-drain electrodes are patterned, (the substrate also functioning as a protective layer), (v) substrate/source-drain electrodes/liquid crystalline organic semiconductor layer/gate insulating layer (which also functions as a liquid crystal alignment layer)/gate electrode/substrate (which also functions as a protective layer), (vi) substrate (which also functions as an alignment layer)/source-drain electrodes/liquid crystalline organic semiconductor layer/gate insulating layer/gate electrode/substrate (which also functions as a protective layer), and (vii) substrate/gate electrode/gate insulating layer/source-drain electrodes/liquid crystalline organic semiconductor layer/substrate (which also functions as an alignment layer).

According to the organic semiconductor device of the invention, its organic semiconductor layer is made of a liquid crystalline organic compound of the invention; therefore, an organic semiconductor thin film uniform and flexible over a wide area can be formed and the film can be applied to a thin film transistor which can be used in a flexible display device or the like, or other devices.

(Process for Producing a Liquid Crystalline Organic Compound)

The process for producing a liquid crystalline organic compound of the invention is a process for producing the above-mentioned liquid crystalline organic compound of the invention, comprising: a step of synthesizing a crude liquid crystalline organic compound; and a step of using at least two kinds of solvents selected from the group consisting of a protonic polar solvent, an aprotonic polar solvent, a basic solvent, a halogenated hydrocarbon solvent, and a nonpolar solvent to purify the synthesized crude liquid crystalline organic compound by a recrystallization method. In the invention, the step of synthesizing a crude liquid crystalline organic compound is varied in accordance with the kind of the liquid crystalline organic compound to be synthesized, and may be performed by, for example, methods in examples which will be described later.

As the mixed solvents for the recrystallization, at least two or more kinds of solvents selected from a protonic polar solvent, an aprotonic polar solvent, a basic solvent, a halogenated hydrocarbon solvent, and a nonpolar solvent are used. The recrystallization method may be recrystallization in which two or more kinds out of the single solvents are successively used, or recrystallization in which two or more kinds out of the single solvents are simultaneously used as mixed solvents. It is particularly preferred that the solvents to be used are solvents in which the solubility of the liquid crystalline organic compound is sufficiently high at high temperature and the solubility is sufficiently low near a room temperature. It is more preferred that the solvents are solvents from which impurities generated in the synthesis process or the like can be removed by the recrystallization. The following will describe examples of the solvents having such property and used for the recrystallization. However, the solvents are not limited to these solvents.

Examples of the protonic polar solvent which can constitute the mixed solvent for the recrystallization include water, methanol, ethanol, 1-propanol, 2-propanol, 1-butanol, and acetic acid. Examples of the aprotonic polar solvent include aromatic hydrocarbon solvents such as toluene, o-xylene, m-xylene, and p-xylene; ketones such as acetone, methyl ethyl ketone, diethyl ketone, and methyl isobutyl ketone; ethers such as diethyl ether, diisopropyl ether, tetrahydrofuran, dioxane, dioxolan, diphenyl ether, and petroleum ether; esters such as methyl formate, ethyl formate, butyl formate, methyl acetate, ethyl acetate, and butyl acetate; amides such as N,N-dimethylformamide, N,N-dimethylacetoamide, N-methyl-2-pyrrolidone, 1,3-dimethyl-2-imidazolidinone, and tetramethylurea; sulfur-containing compounds such as dimethylsulfoxide, sulfolane, and hexamethylphosphoroamide; and acetonitrile. Examples of the basic solvent include pyridine, α-picoline, 2,6-lutidine, and N-methylmorpholine. Examples of the halogenated hydrocarbon solvent include o-chlorobenzene, m-chlorobenzene, p-chlorobenzene, dichlorobenzene, methyl chloride, chloroform, and carbon tetrachloride. Examples of the nonpolar solvent include benzene, cyclohexane, pentane, hexane, heptane, octane, and pentene. In the invention, two or more kinds out of these solvents are used, whereby impurities having different solubilities can be dissolved in at least two kinds selected from the protonic polar solvent, the aprotonic polar solvent, the basic solvent, the halogenated hydrocarbon solvent, and the nonpolar solvent. As a result, in this case, the recrystallization can be more effectively attained than in the case of subjecting the crude compound to multiple recrystallization using only one kind of solvent.

Particularly preferred recrystallizing solvents out of these solvents are hexane (a nonpolar solvent), ethanol (a protonic polar solvent), acetone (an aprotonic polar solvent), and others.

The recrystallization can be performed in a known manner. The crystal obtained by the recrystallization is filtrated, washed, and then dried in an appropriate manner. In the step of the recrystallization, the liquid crystalline organic compound may be caused to contact an absorbent, whereby the amount of impurities can be made smaller. Preferred examples of the method therefor include a batch method of adding the absorbent into the solvent, and then stirring the solution, and a flow method of causing the solution to pass into a layer of the absorbent filled into a column. As the absorbent, activated carbon, alumina, silica, zeolite or the like is preferably used. Activated carbon is particularly preferred.

According to the process for producing a liquid crystalline organic compound of the invention, a crude synthesized liquid crystalline organic compound is purified by a recrystallization method using at least two kinds of solvents selected from the group consisting of a protonic polar solvent, an aprotonic polar solvent, a basic solvent, a halogenated hydrocarbon solvent, and a nonpolar solvent; therefore, impurities which cause a fall in the charge mobility can be effectively removed. As a result, it is possible to produce effectively a liquid crystalline organic compound which gives a liquid crystal phase in a temperature range containing at least a room temperature and can exhibit a high charge mobility.

### EXAMPLES

The invention will be more specifically described by way of the following examples. However, the invention is not limited to these examples.

### Example 1

### Synthesis of 2-(4′-heptyloxyphenyl)-6-dodecylbenzothiazole (abbreviated to 70-PBT-12 hereinafter)

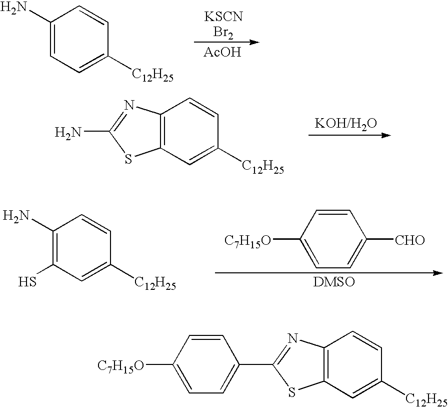

Acetic acid (900 ml) was added to 4-dodecylaniline (60 g, 229.4 mmol) and potassium thiocyanate (55.7 g, 573.8 mmol), and the resultant solution was stirred at 25° C. for 1 hour. While the solution was cooled in an ice bath, a solution of bromine in acetic acid (Br₂/AcOH=14.1 ml/150 ml) was dropwise added thereto, and the resultant was stirred at 25° C. for 24 hours. After the stirring, hot water (500 ml) was added to the solution, and the resultant was filtrated. Thereafter, ammonium water was added to the filtrate until the solution turned alkaline. The precipitated solid was extracted with ethyl acetate, and further isolated with a column chromatography (developing solvent was hexane:ethyl acetate=1:2). Thereafter, the solid was recrystallized from ethyl acetate to yield 43.8 g of 2-amino-6-dodecylbenzothiazole (yield: 41.8%).

Next, water (100 ml) was added to 2-amino-6-dodecylbenzothiazole (10 g, 31 mmol) and potassium hydroxide (86.9 g, 1550 mmol), and the resultant solution was stirred for 15 hours while refluxed. The content was filtrated and diluted with cold water. Thereto was added 5 N of acetic acid so as to neutralize the solution. The precipitated solid was extracted with diisopropyl ether to yield 8.49 g of 2-amino-5-dodecylthiophenol (yield: 93.2%).

Furthermore, in a solvent (20 ml) of DMSO (dimethylsulfoxide), 2-amino-5-dodecylthiophenol (4 g, 13.6 mmol) and p-heptyloxybenzaldehyde (3 g, 13.6 mmol) were stirred at 150° C. for 3 hours. After the end of the reaction, the temperature of the solution was returned to the room temperature, and then water was added to the solution. The solution was filtrated. The obtained solid was extracted with ethyl acetate and further isolated with a column chromatography (developing solvent:hexane). Thereafter, the solid was recrystallized from acetone to yield 3.36 g of 70-PBT-12 (yield 50%).

About the resultant 70-PBT-12, phase transition temperatures thereof were measured by DSC (differential scanning calorimetry). As a result, under temperature-lowering conditions, the following result was obtained: “Iso (isotropic phase)/97.3° C./SmA (smectic A phase)/42.6° C./Cryst (crystal phase)”. The temperature between any two of these phases shows one out of the phase transition temperatures. Identification of the liquid crystal phase was performed by X-ray diffraction.

As a liquid crystal cell, there was used a glass substrate having ITO electrodes (surface resistance: 100 to 200 Ω/□) formed by vacuum film-formation and having a cell gap of 15 μm. The sample was purified by recrystallization using hexane, ethanol and acetone in this order. The sample was injected into the liquid crystal cell by infiltrating the sample heated until the sample exhibited an isotropic phase into the liquid crystal cell heated onto a hot plate, using a capillary phenomenon. The mobility was measured in the liquid crystal phase of the sample by the TOF method using a light source emitting a nitrogen laser having a wavelength of 337 nm. At this time, the mobility of holes in the SmA was 1.5×10⁻³ cm²/V·s or more.

### Example 2

### Synthesis of 2-(4′-octylphenyl)-6-butoxybenzothiazole (abbreviated to 8-PBT-04 hereinafter)

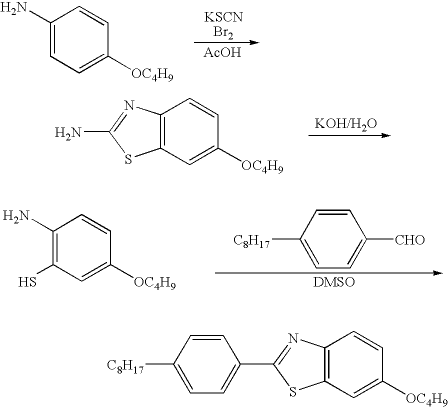

Acetic acid (200 ml) was added to 4-butoxyaniline (25 g, 151 mmol) and potassium thiocyanate (36.7 g, 378 mmol), and the resultant solution was stirred at 25° C. for 1 hour. While the solution was cooled in an ice bath, a solution of bromine in acetic acid (Br₂/AcOH=9.3 ml/50 ml) was dropwise added thereto, and the resultant was stirred at 25° C. for 24 hours. After the stirring, hot water (125 ml) was added to the solution, and the resultant was filtrated. Thereafter, ammonium water was added to the filtrate until the solution turned alkaline. The precipitated solid was extracted with ethyl acetate, and further isolated with a column chromatography (developing solvent was hexane: ethyl acetate=1:2). Thereafter, the solid was recrystallized from ethyl acetate to yield 17.1 g of 2-amino-6-butoxybenzothiazole (yield: 51%).

Next, water (50 ml) was added to 2-amino-6-butoxybenzothiazole (5 g, 22.4 mmol) and potassium hydroxide (62 g, 1120 mmol), and the resultant solution was stirred for 15 hours while refluxed. The content was filtrated and diluted with cold water. Thereto was added 5 N of acetic acid so as to neutralize the solution. The precipitated solid was extracted with diisopropyl ether to yield 3.7 g of 2-amino-5-butoxythiophenol (yield: 83.4%).

Furthermore, in a solvent (25 ml) of DMSO (dimethylsulfoxide), 2-amino-5-butoxythiophenol (2.5 g, 12.6 mmol) and p-octylbenzaldehyde (2.75 g, 12.6 mmol) were stirred at 150° C. for 3 hours. After the end of the reaction, the temperature of the solution was returned to the room temperature, and then water was added to the solution. The solution was filtrated. The obtained solid was extracted with ethyl acetate and further isolated with a column chromatography (developing solvent: hexane). Thereafter, the solid was recrystallized from acetone to yield 2.5 g of 8-PBT-04 (yield: 50%).

About the resultant 8-PBT-04, phase transition temperatures thereof were measured by DSC (differential scanning calorimetry). As a result, under temperature-lowering conditions, the following result was obtained: “Iso/84.8° C./SmA/20.3° C./Cryst”. Identification of the liquid crystal phase was performed by X-ray diffraction.

As a liquid crystal cell, there was used a glass substrate having ITO electrodes (surface resistance: 100to200 Ω/□) formed by vacuum film-formation and having a cell gap of 15 μm. The sample was purified by recrystallization using hexane, ethanol and acetone in this order. The sample was injected into the liquid crystal cell by infiltrating the sample heated until the sample exhibited an isotropic phase into the liquid crystal cell heated onto a hot plate, using a capillary phenomenon. The mobility was measured in the liquid crystal phase of the sample by the TOF method using a light source emitting a nitrogen laser having a wavelength of 337 nm. At this time, the mobility of holes in the SmA was 1.0×10⁻³ cm²/V·s or more.

### Example 3

### Synthesis of 2-(4′-octylphenyl)-6-dodecylbenzothiazole (abbreviated to 8-PBT-12 hereinafter)

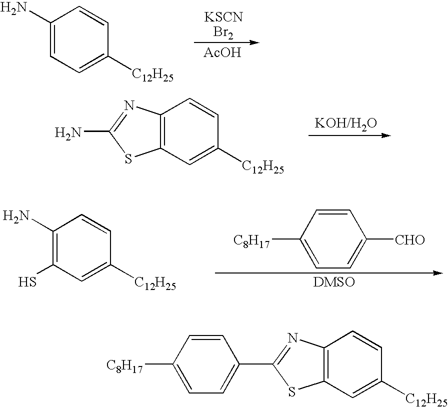

Acetic acid (900 ml) was added to 4-dodecylaniline (60 g, 229.4 mmol) and potassium thiocyanate (55.7 g, 573.8 mmol), and the resultant solution was stirred at 25° C. for 1 hour. While the solution was cooled in an ice bath, a solution of bromine in acetic acid (Br₂/AcOH=14.1 ml/150 ml) was dropwise added thereto, and the resultant was stirred at 25° C. for 24 hours. After the stirring, hot water (500 ml) was added to the solution, and the resultant was filtrated. Thereafter, ammonium water was added to the filtrate until the solution turned alkaline. The precipitated solid was extracted with ethyl acetate, and further isolated with a column chromatography (developing solvent was hexane:ethyl acetate=1:2). Thereafter, the solid was recrystallized from ethyl acetate to yield 43.8 g of 2-amino-6-dodecylbenzothiazole (yield: 41.8%).

Next, water (100 ml) was added to 2-amino-6-dodecylbenzothiazole (10 g, 31 mmol) and potassium hydroxide (86.9 g, 1550 mmol), and the resultant solution was stirred for 15 hours while refluxed. The content was filtrated and diluted with cold water. Thereto was added 5 N of acetic acid so as to neutralize the solution. The precipitated solid was extracted with diisopropyl ether to yield 8.49 g of 2-amino-5-dodecylthiophenol (yield: 93.2%).

Furthermore, in a solvent (20 ml) of DMSO (dimethylsulfoxide), 2-amino-5-dodecylthiophenol (2.3 g, 10.5 mmol) and p-octylbenzaldehyde (2.3 g, 10.5 mmol) were stirred at 150° C. for 3 hours. After the end of the reaction, the temperature of the solution was returned to the room temperature, and then water was added to the solution. The solution was filtrated. The obtained solid was extracted with ethyl acetate and further isolated with a column chromatography (developing solvent: hexane). Thereafter, the solid was recrystallized from acetone to yield 2.5 g of 8-PBT-12 (yield: 48.4%).

Phase transition temperatures thereof were measured by DSC (differential scanning calorimetry). As a result, under temperature-lowering conditions, the following result was obtained: “Iso/71.4° C./SmA/44.9° C./SmB/about −50° C.”. The wording “about −50° C.” herein means that the lower limit of the measurement by the DSC is −50° C., and at this lower measurement limit the phase transition temperature, at which the compound 8-PBT-12 undergoes crystal transition, was unable to be found out. Identification of the liquid crystal phase was performed by X-ray diffraction.

As a liquid crystal cell, there was used a cell obtained by coating titanium oxide into a thickness of 250 nm onto a glass substrate, on which an ITO electrode (surface resistance: 100 to 200 Ω/□) was formed by vacuum film-formation, by spin coating and further causing the substrate to adhere onto a glass substrate, on which an ITO electrode (surface resistance: 100 to 200 Ω/□) was formed by vacuum film-formation, through a spacer having a thickness of 15 μm. The sample was purified by recrystallization using hexane, ethanol and acetone in this order. The sample was injected into the liquid crystal cell by infiltrating the sample heated until the sample exhibited an isotropic phase into the liquid crystal cell heated onto a hot plate, using a capillary phenomenon. The mobility was measured in the liquid crystal phase of the sample by the TOF method using a light source emitting a nitrogen laser having a wavelength of 337 nm. At this time, the mobility of holes in the SmB was 1.0×10⁻² cm²/V·s or more.

### Example 4

### Synthesis of 6,6′-bisdodecyl-2,2′-bibenzothiazole (abbreviated to 12-TB-BT-12 hereinafter)

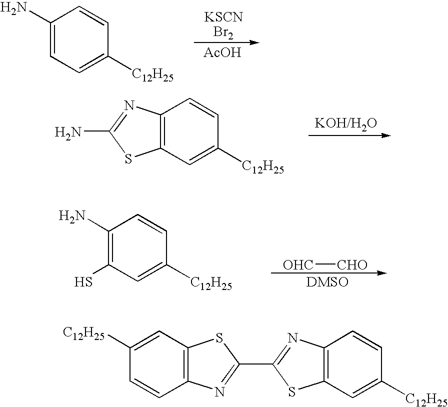

Acetic acid (900 ml) was added to 4-dodecylaniline (60 g, 229.4 mmol) and potassium thiocyanate (55.7 g, 573.8 mmol), and the resultant solution was stirred at 25° C. for 1 hour. While the solution was cooled in an ice bath, a solution of bromine in acetic acid (Br₂/AcOH=14.1 ml/150 ml) was dropwise added thereto, and the resultant was stirred at 25° C. for 24 hours. After the stirring, hot water (500 ml) was added to the solution, and the resultant was filtrated. Thereafter, ammonium water was added to the filtrate until the solution turned alkaline. The precipitated solid was extracted with ethyl acetate, and further isolated with a column chromatography (developing solvent was hexane:ethyl acetate=1:2). Thereafter, the solid was recrystallized from ethyl acetate to yield 43.8 g of 2-amino-6-dodecylbenzothiazole (yield: 41.8%).

Next, water (100 ml) was added to 2-amino-6-dodecylbenzothiazole (10 g, 31 mmol) and potassium hydroxide (86.9 g, 1550 mmol), and the resultant solution was stirred for 15 hours while refluxed. The content was filtrated and diluted with cold water. Thereto was added 5 N of acetic acid so as to neutralize the solution. The precipitated solid was extracted with diisopropyl ether to yield 8.49 g of 2-amino-5-dodecylthiophenol (yield: 93.2%).

Furthermore, in a solvent (15 ml) of DMSO (dimethylsulfoxide), 2-amino-5-dodecylthiophenol (3 g, 10.2 mmol) and a glyoxal solution (0.73 g, 12.5 mmol) were stirred at 150° C. for 3 hours. After the end of the reaction, the temperature of the solution was returned to the room temperature, and then water was added to the solution. The solution was filtrated. The obtained solid was extracted with ethyl acetate and further isolated with a column chromatography (developing solvent: hexane). Thereafter, the solid was recrystallized from toluene to yield 3.1 g of 12-TB-BT-12 (yield: 51%). Identification of the liquid crystal phase was performed by X-ray diffraction.

As a liquid crystal cell, there was used a glass substrate having ITO electrodes (surface resistance: 100 to 200 Ω/□) formed by vacuum film-formation and having a cell gap of 15 μm. The sample was purified by recrystallization using hexane, ethanol and acetone in this order. The sample was injected into the liquid crystal cell by infiltrating the sample heated until the sample exhibited an isotropic phase into the liquid crystal cell heated onto a hot plate, using a capillary phenomenon. The mobility was measured in the liquid crystal phase of the sample by the TOF method using a light source emitting a nitrogen laser having a wavelength of 337 nm. At this time, the mobility of holes in the liquid crystal phase was 1.5×10⁻³ cm²/V·s or more.

### Example 5

### Synthesis of 6-dodecyl-2-(2′-heptylbenzothiazole-6′-yl)benzothiazole (abbreviated to 7-BT-BT-12 hereinafter)

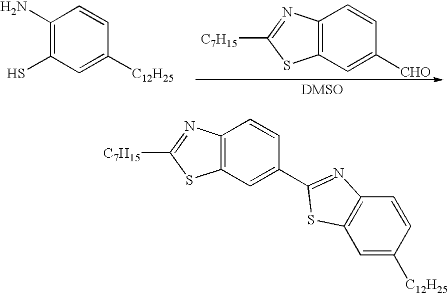

In a solvent (20 ml) of DMSO (dimethylsulfoxide), 2-amino-5-dodecylthiophenol (4 g, 13.6 mmol) and 2-heptyl-6-formylbenzothiazole (3.6 g, 13.6 mmol) were stirred at 150° C. for 3 hours. After the end of the reaction, the temperature of the solution was returned to the room temperature, and then water was added to the solution. The solution was filtrated. The obtained solid was extracted with toluene and further isolated with a column chromatography (developing solvent: toluene). Thereafter, the solid was recrystallized from toluene to yield 3.2 g of 7-BT-BT-12 (yield: 41%).

As a liquid crystal cell, there was used a glass substrate having ITO electrodes (surface resistance: 100 to 200 Ω/□) formed by vacuum film-formation and having a cell gap of 15 μm. The sample was purified by recrystallization using hexane, ethanol and acetone in this order. The sample was injected into the liquid crystal cell by infiltrating the sample heated until the sample exhibited an isotropic phase into the liquid crystal cell heated onto a hot plate, using a capillary phenomenon. The mobility was measured in the liquid crystal phase of the sample by the TOF method using a light source emitting a nitrogen laser having a wavelength of 337 nm. At this time, the mobility of holes in the liquid crystal phase was 2.0×10⁻³ cm²/V·s or more.

### Example 6

### Synthesis of2,2′-bispentyl-6,6′-bibenzothiazole (abbreviated to 7-BT-TB-7 hereinafter)

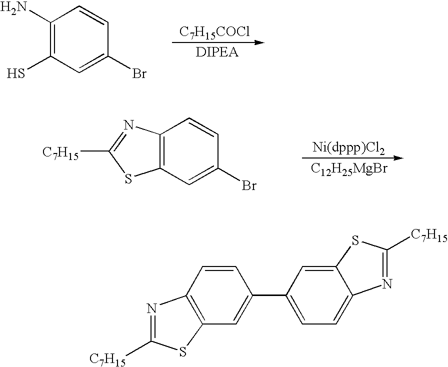

In a solvent (30 ml) of diisopropylethylamine (abbreviated to DIPEA hereinafter), 2-amino-5-bromothiophenol (3 g, 14.6 mmol) and heptynoyl chloride (2.4 g, 14.6 mmol) were caused to react with each other to yield 2.37 g of 2-heptyl-6-bromobenzothiazole (yield: 52%). Next, in a diethyl ether solvent (50 ml), 2-heptyl-6-bromobenzothiazole (2 g, 6.4 mmol) was caused to react with a Grignard reagent (dodecylbromomagnesium (1.75 g, 6.4 mmol)) in the presence of a [1,3-bis(diphenylphosphino)propane]nickel (II) chloride catalyst (0.35 g, 0.64 mmol) at 40° C. for 24 hours. Thereafter, the reaction solution was extracted with toluene. The obtained solid was further isolated with a column chromatography (developing solvent: toluene) and recrystallized from toluene to yield 1.43 g of 7-BT-TB-7 (yield: 48%). Identification of the liquid crystal phase was performed by X-ray diffraction.

As a liquid crystal cell, there was used a glass substrate having ITO electrodes (surface resistance: 10 0to 200 Ω/□) formed by vacuum film-formation and having a cell gap of 15 μm. The sample was purified by recrystallization using hexane, ethanol and acetone in this order. The sample was injected into the liquid crystal cell by infiltrating the sample heated until the sample exhibited an isotropic phase into the liquid crystal cell heated onto a hot plate, using a capillary phenomenon. The mobility was measured in the liquid crystal phase of the sample by the TOF method using a light source emitting a nitrogen laser having a wavelength of 337 nm. At this time, the mobility of holes in the liquid crystal phase was 2.0×10⁻³ cm²/V·s or more.

### Comparative Example 1

### Synthesis of 2-(4′-heptyloxyphenyl)-6-dodecylthiobenzothiazole (abbreviated to 70-PBT-S12 hereinafter)

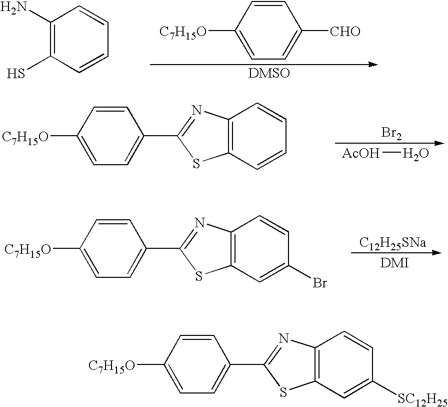

In a solvent (100 ml) of DMSO (dimethylsulfoxide), 1,2-aminobenzenethiol (10 g, 79.8 mmol) and p-heptyloxybenzaldehyde (17.6 g, 79.8 mmol) were stirred at 150° C. for 3 hours. After the end of the reaction, the temperature of the solution was returned to the room temperature, and then water was added to the solution. The solution was filtrated. The obtained solid was extracted with ethyl acetate and further isolated with a column chromatography (developing solvent: hexane). Thereafter, the solid was recrystallized from hexane to yield 15.3 g of 2-(4′-heptyloxyphenyl)benzothiazole (yield: 59%).

Next, to a solvent (200 ml) of acetic acid was added 2-(4′-heptyloxyphenyl)benzothiazole (15 g, 46.1 mmol), and subsequently thereto was dropwise added a solution of bromine in acetic acid (Br₂/AcOH=11.2 ml/125 ml). The resultant solution was stirred at 25° C. for 24 hours. After the end of the reaction, the reactant product was extracted with ethyl acetate and then isolated with a column chromatography (developing solvent: hexane). Thereafter, the solid was recrystallized from hexane to yield 8.9 g of 6-bromo-2-(4′-heptyloxyphenyl)benzothiazole (yield: 48%).

Finally, in a solvent (50 ml) of N,N-dimethylimidazolidinone (DMI), 6-bromo-2-(4′-n-heptyloxyphenyl)benzothiazole (5 g, 12.4 mmol) was caused to react with a sodium salt (2.78 g, 12.4 mmol) of dodecylthiol at 60° C. for 24 hours. After the end of the reaction, the reactant product was extracted with ethyl acetate and then isolated with a column chromatography (developing solvent: hexane). Thereafter, the solid was recrystallized from acetone to yield 3.7 g of 70-PBT-S12 (yield: 56%).

Phase transition temperatures thereof were measured by DSC (differential scanning calorimetry). As a result, under temperature-lowering conditions, the following result was obtained: “Iso/100° C./SmA/90° C./Cryst”. Identification of the liquid crystal phase was performed by X-ray diffraction.

As a liquid crystal cell, there was used a glass substrate having ITO electrodes (surface resistance: 100 to 200 Ω/□) formed by vacuum film-formation and having a cell gap of 15 μm. The sample was purified by recrystallization using hexane, ethanol and acetone in this order. The sample was injected into the liquid crystal cell by infiltrating the sample heated until the sample exhibited an isotropic phase into the liquid crystal cell heated onto a hot plate, using a capillary phenomenon.

The mobility was measured in the SmA phase by the TOF method using a light source emitting a nitrogen laser having a wavelength of 337 nm. However, in the resultant product, a byproduct in which the 3′ position of the phenylbenzothiazole derivative was brominated was produced, and the target product was not easily purified. This product has a carbon-sulfur bond, but the carbon-sulfur bond is easily oxidized, so that the product is chemically instable and is not easily purified. The electric conduction thereof was mainly dominated by ionic conduction, and the mobility of holes was 1.0×10⁻⁶ cm²/V·s or less.

### Comparative Example 2

### Synthesis of 2-(4′-heptyloxyphenyl)-6-dodecylbenzothiazole (abbreviated to 70-PBT-12 hereinafter)

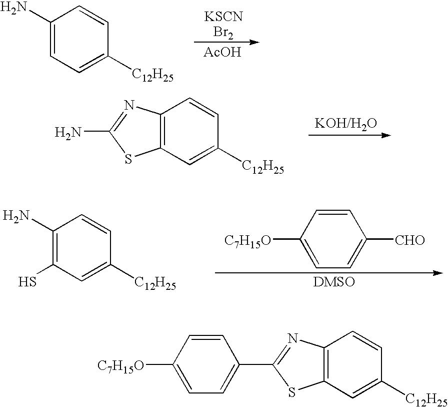

Acetic acid (900 ml) was added to 4-dodecylaniline (60 g, 229.4 mmol) and potassium thiocyanate (55.7 g, 573.8 mmol), and the solution was stirred at 25° C. for 1 hour. While the solution was cooled in an ice bath, thereto was dropwise added a solution of bromine in acetic acid (Br₂/AcOH=14.1 ml/150 ml). The resultant solution was stirred at 25° C. for 24 hours. After the stirring, hot water (500 ml) was added to the solution, and then the resultant solution was filtrated. Thereafter, ammonia water was added to the filtrate until the solution turned alkaline. The precipitated solid was extracted with ethyl acetate and then isolated with a column chromatography (developing solvent was hexane:acetic acid=1:2). Thereafter, the solid was recrystallized from ethyl acetate to yield 43.8 g of 2-amino-6-dodecylbenzothiazole (yield: 41.8%).

Next, water (100 ml) was added to 2-amino-6-dodecylbenzothiazole (10 g, 31 mmol) and potassium hydroxide (86.9 g, 1550 mmol). The solution was stirred for 15 hours while refluxed. The content was filtrated, and diluted with cold water. Thereto was added 5 N of acetic acid so as to neutralize the solution. The precipitated solid was extracted with diisopropyl ether to yield 8.49 g of 2-amino-5-dodecylthiophenol (yield: 93.2%).

Furthermore, in a solvent (20 ml) of DMSO (dimethylsulfoxide), 2-amino-5-dodecylthiophenol (4 g, 13.6 mmol) and p-heptyloxybenzaldehyde (3 g, 13.6 mmol) were stirred at 150° C. for 3 hours. After the end of the reaction, the temperature of the solution was returned to the room temperature, and then water was added to the solution. The solution was filtrated. The obtained solid was extracted with ethyl acetate and further isolated with a column chromatography (developing solvent: hexane). Thereafter, the solid was recrystallized from acetone to yield 3.36 g of 70-PBT-12 (yield: 50%).

Phase transition temperatures thereof were measured by DSC (differential scanning calorimetry). As a result, under temperature-lowering conditions, the following result was obtained: “Iso/97.3° C./SmA/42.6° C./Cryst”. Identification of the liquid crystal phase was performed by X-ray diffraction.

As a liquid crystal cell, there was used a glass substrate having ITO electrodes (surface resistance: 100 to 200 Ω/□) formed by vacuum film-formation and having a cell gap of 15 μm. The sample was purified by recrystallization using only hexane. The sample was injected into the liquid crystal cell by infiltrating the sample heated until the sample exhibited an isotropic phase into the liquid crystal cell heated onto a hot plate, using a capillary phenomenon.

The mobility was measured in the SmA phase of the sample by the TOF method using a light source emitting a nitrogen laser having a wavelength of 337 nm. However, impurities were not removed by the recrystallization using only hexane, and the product was not easily purified. The electric conduction thereof was mainly dominated by ionic conduction, and the mobility of holes was 1.5×10⁻⁶ cm²/V·s or less.

(Evaluating Method)

The samples synthesized in Examples 1 to 6 and Comparative Examples 1 and 2 were tested about the following evaluating items. Data therefrom were measured.

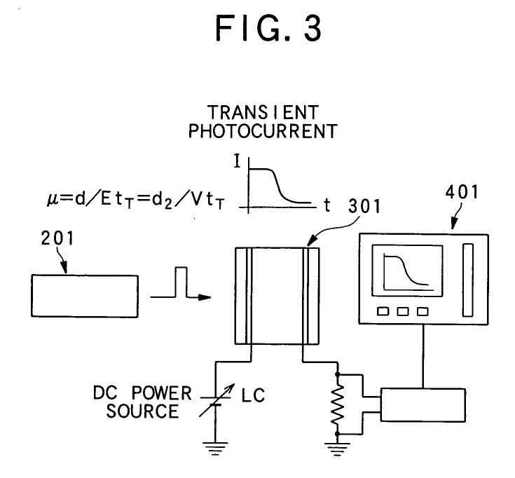

For the measurement of the phase transition temperatures, a DSC (differential scanning calorimetry, DSC220C, manufactured by Seiko Instruments Inc.) was used. For the identification of the liquid crystal phases, an X-ray diffraction device with a hot stage (RIGAKU RAD-B, manufactured by Rigaku Corporation) was used. For the measurement of the charge mobilities, a device shown in FIG. 3 was used, and a nitrogen pulse maser (Laser photonics LN203C) was used as exciting light. In the liquid crystal phase of each of the samples, the charge mobility was measured by the TOF (Time Of Flight) method.

TABLE 3

| | Charge mobility |
|---|---|
| Example 1 | 1.5 × 10⁻³ cm²/V · s or more |
| Example 2 | 1.0 × 10⁻³ cm²/V · s or more |
| Example 3 | 1.0 × 10⁻² cm²/V · s or more |
| Example 4 | 1.5 × 10⁻³ cm²/V · s or more |
| Example 5 | 2.0 × 10⁻³ cm²/V · s or more |
| Example 6 | 2.0 × 10⁻³ cm²/V · s or more |
| Comparative Example 1 | 1.0 × 10⁻⁶ cm²/V · s or less |
| Comparative Example 2 | 1.5 × 10⁻⁶ cm²/V · s or less |

According to Table 3, in the case of using a benzothiazole ring in the core of a liquid crystalline organic compound and combining the 2 position and the 2 position, the 2 position and the 6 position, or the 6 position and the 6 position of benzothiazole rings, the charge mobility was improved. On the other hand, when a carbon-sulfur bond was present in a spacer portion of a phenylbenzothiazole derivative as seen in Comparative Example 1, the derivative was easily oxidized and was chemically instable so as not to be easily purified. The electric conduction thereof was mainly dominated by ionic conduction, and the mobility was not improved.

It is understood from comparison of Example 1 with Comparative Example 2 that the recrystallization using two or more kinds out of the specific solvents as seen in Example 1 produces a larger effect on the purification than the recrystallization using a single solvent as seen in Comparative Example 2.

What is claimed is:

1. A liquid crystalline organic compound, wherein any one of a benzothiazole skeleton, a benzoselenazole skeleton, a benzoxazole skeleton and an indene skeleton represented by a following chemical formula 1: 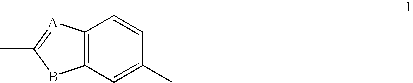
in which A is a nitrogen atom or a CH group, and B is a sulfur atom, a selenium atom or an oxygen atom, is contained as Z1 in a following chemical formula 2:
R1-Y1-Z1-Y2-R2 2
in which R1 and R2 are each independently a saturated hydrocarbon or an unsaturated hydrocarbon of a straight chain structure, a branched chain structure or a cyclic structure having 1 to 22 carbon atoms; R1 may be bonded directly to Z1 without interposing Y1 therebetween and R2 may be bonded directly to Z1 without interposing Y2 therebetween; and Y1 and Y2 are each independently selected from a group consisting of the oxygen atom and the selenium atom and a —CO— group, a —OCO-group, a —COO— group, a —N═CH— group, a —CONH— group, a —NH— group, a —NHCOO group and a —CH₂ group.

2. A liquid crystalline organic compound, wherein any one of a benzothiazole skeleton, a benzoselenazole skeleton, a benzoxazole skeleton and an indene skeleton represented by a following chemical formula 1: 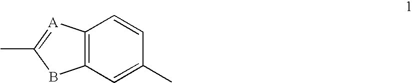
in which A is a nitrogen atom or a CH group, and B is a sulfur atom, a selenium atom or an oxygen atom, is contained as each of Z1 and Z2 in a following chemical formula 3:
R1-Y1-Z1-X-Z2-Y2-R2 3
in which R1 and R2 are each independently a saturated hydrocarbon or an unsaturated hydrocarbon of a straight chain structure, a branched chain structure or a cyclic structure having 1 to 22 carbon atoms; R1 may be bonded directly to Z1 without interposing Y1 therebetween and R2 may be bonded directly to Z2 without interposing Y2 therebetween; Y1 and Y2 are each independently selected from a group consisting of an oxygen atom and a selenium atom and a —CO— group, a —OCO— group, a —COO— group, a —N═CH— group, a —CONH— group, a —NH— group, a —NHCOO group and a —CH₂ group; and X may be a saturated hydrocarbon or an unsaturated hydrocarbon having a straight chain structure, a branched chain structure or a cyclic structure having 1 to 22 carbon atoms, or X may not be present so as to bond Z1 directly to Z2.

3. The liquid crystalline organic compound according to claim 1, wherein at least one kind of smectic liquid crystal phase state at a pyrolysis temperature thereof or lower is exhibited.

4. The liquid crystalline organic compound according to claim 2, wherein at least one kind of smectic liquid crystal phase state at a pyrolysis temperature thereof or lower is exhibited.

5. An organic semiconductor structure having an organic semiconductor layer comprising the liquid crystalline organic compound according to claim 1,
wherein the liquid crystalline organic compound exhibits at least one kind of smectic liquid crystal phase state at the pyrolysis temperature thereof or lower, and the organic semiconductor layer has an electron mobility of 1.0×10⁻⁵ cm²/V·s or more, or a hole transporting mobility of 1.0×10⁻⁵ cm²/V·s or more.

6. An organic semiconductor structure having an organic semiconductor layer comprising the liquid crystalline organic compound according to claim 2,
wherein the liquid crystalline organic compound exhibits at least one kind of smectic liquid crystal phase state at the pyrolysis temperature thereof or lower, and the organic semiconductor layer has an electron mobility of 1.0×10⁻⁵ cm²/V·s or more, or a hole transporting mobility of 1.0×10⁻⁵ cm²/V·s or more.

7. An organic semiconductor device having at least a substrate, a gate electrode, a gate insulating layer, an organic semiconductor layer, a drain electrode, and a source electrode,
wherein the organic semiconductor layer is formed to comprise the liquid crystalline organic compound according to claim 1.

8. An organic semiconductor device having at least a substrate, a gate electrode, a gate insulating layer, an organic semiconductor layer, a drain electrode, and a source electrode,
wherein the organic semiconductor layer is formed to comprise the liquid crystalline organic compound according to claim 2.

9. A process for producing the liquid crystalline organic compound according to claim 1, comprising:
a step of synthesizing a crude liquid crystalline organic compound; and
a step of using at least two kinds of solvents selected from a group consisting of a protonic polar solvent, an aprotonic polar solvent, a basic solvent, a halogenated hydrocarbon solvent, and a nonpolar solvent to purify the synthesized crude liquid crystalline organic compound by a recrystallization method.

10. A process for producing the liquid crystalline organic compound according to claim 2, comprising:
a step of synthesizing a crude liquid crystalline organic compound; and
a step of using at least two kinds of solvents selected from a group consisting of a protonic polar solvent, an aprotonic polar solvent, a basic solvent, a halogenated hydrocarbon solvent, and a nonpolar solvent to purify the synthesized crude liquid crystalline organic compound by a recrystallization method.
